(19) United States
(12) Patent Application Publication
(10) Pub. No.: US 2022/0249347 A1
(43) Pub. Date: Aug. 11, 2022

(54) Composition and Method Containing Pea and Quinoa Natural Peptides And Niacinamide to Enhance Skin Glow, Radiance And Even Skin Tone

(71) Applicant: TRI-K Industries, Inc., Denville, NJ (US)

(72) Inventors: Pushker Sona, Denville, NJ (US); Dr. Mihaela Gorcea, Denville, NJ (US); Svetlana Gorelik, Denville, NJ (US)

(73) Assignee: TRI-K Industries, Inc., Denville, NJ (US)

(21) Appl. No.: 17/669,136

(22) Filed: Feb. 10, 2022

Related U.S. Application Data
(60) Provisional application No. 63/147,868, filed on Feb. 10, 2021.

(51) Int. Cl. A61K 8/64; A61K 8/67; A61Q 19/02

(52) U.S. Cl. CPC A61K 8/645; A61K 2800/524; A61Q 19/02; A61K 8/675

(57) ABSTRACT

Healthy glow is characterized by radiant skin, brighter, lighter, even skin tone and clear skin without blemishes. Skin pigmentation is a process known as melanogenesis involving production, transportation, and distribution of melanin from the epidermis's inner layer to the skin's surface. There are multiple biological processes and underlying gene pathways that impact overall melanogenesis. Disclosed is a composition and methods for a combination of Vitamin B3 and Pea and Quinoa peptides obtained from hydrolyzed proteins. This composition was found to offer a holistic approach to the skin glow through multi targeted action controlling key biological pathways. Vitamin B3, Pea peptides, and Quinoa peptides were found to deliver a synergistic effect for desired skin glow, radiance, even skin tone, uniformity, homogeneity, smoothing skin texture, reducing dark spots/hyperpigmentation and improve skin appearance without any reported side effects as compared to formulations without the composition.

### CROSS REFERENCE TO RELATED APPLICATION

This application claims priority from U.S. provisional application No. 63/147,868 filed on Feb. 10, 2021, the disclosure of which is hereby incorporated herein by reference.

### FIELD OF USE

The present application relates to the field of cosmetics, and skin health care. Specifically, the present application discloses a method and a composition formulation that contains blends of various natural peptides with niacinamide to form a compound that has unexpectedly profound skin care effects and controls skin pigmentation through a multi-targeted biological mechanism of action. This novel method and composition of two natural peptides and niacinamide provides benefits which address variety of skin appearance benefits including skin pigmentation improving skin glow, reducing dark spots/hyperpigmentation, evening skin tone, and improving radiance.

### BACKGROUND OF THE INVENTION

A healthy skin glow is ever evolving consumer need characterized by skin that is radiant, even skin tone and clear skin without blemishes. Vitamins like B3 (niacinamide) has been used for long in the industry to control the skin pigmentation through a robust mechanism of action. Skin pigmentation however is complex. A process, known as melanogenesis, affects skin pigmentation. This process involves production, transportation, and distribution of melanin from the inner layer of the epidermis to the outer layer at skin's surface. There are multiple biological processes and underlying gene pathways that impacts the overall melanogenesis. By upregulating or down regulating certain pathways, one can carefully control the end to end process. Proteins and peptides are fundamental building blocks of skin and have potency to regulate a variety of healthy skin pathways including pigmentation.

Skin lightening products provide solutions to different problems addressing specific consumer requirements related to spots, blemishes, pigmentation, and other related issues. Shifting trend towards skin lightening in the Asian and African countries as a matter of enhanced beauty has increased the prominence of these products in these regions. The primary focus of lightening- is all about reducing pigmentation, dark spots, acne spots, suntan and lighten the specific areas to even out skin tone.

Amongst many skin lightening actives used in the industry such as kojic acid, arbutin, hydroxy acids and variety of natural ingredients, niacinamide is universally recognized as most safe and proven technology. Niacinamide is also used widely across mainstay and masstige brands globally. Despite advancement in pigmentation research, there remains a gap in meeting ever growing need for superior skin tone management and compelling proposition around glow, radiance, and even skin tone.

In addition, there is an unmet need due to the ongoing growth of skin lightening category and recent trends of skin glow within skin care. A superior technology is needed to combine a differentiating proposition around glow based on niacinamide. Combining niacinamide with substances such as natural peptides has many technological challenges. Niacinamide is a known for skin flushing/tingling sensation/itching, or redness which can give some skin discomfort to consumers.

Current state of the art compositions with niacinamide alone still fail to offer effective and holistic skin appearance benefits including glow for various types of skin. Many companies use a variety of complementary technologies which may either be too expensive, or not fully safe and efficacious. The industry yearns for safe and effective solutions for delivering consumer this unmet need of glowing skin. Therefore, there still exists a critical need in the art for an effective composition that is healthy and enhanced at an affordable cost.

### SUMMARY OF THE INVENTION

Compared to the above prior attempts, the presently disclosed methods and composition solves the problems of current state of the art, meets the above requirements, and provides many more benefits. The present disclosure is a unique composition and methods for skin healthcare and provides many benefits.

Incorporation of natural peptides are generally used to nourish, moisturize, and improve the health of skin. This novel composition of two natural peptides and niacinamide provides benefits which address skin pigmentation including improving skin glow, reducing dark spots/hyperpigmentation, evening skin tone, and improving radiance.

The powerful combination of Vitamin B3 (niacinamide) and natural peptides therefore can offer a holistic approach to the skin glow through multi targeted action controlling key biological pathways. Therefore, it is proposed a technology combining the power of Vitamin B3 and an optimum combination of natural peptides to deliver the desired end benefit of enhancing skin glow, radiance and even skin tone.

In one aspect, the present invention relates to improvement of skin glow, radiance, even skin tone, even complexion, reduction of the dark spots and hyperpigmentation, and overall improvement of skin's visual appearance and general health. Depending on the embodiment, the composition contains the combination, at an optimal ratio, of two complementary natural peptides ingredients, Pea and Quinoa, obtained from hydrolyzation of Pea and Quinoa proteins with the niacinamide to provide an enhanced skin brightening, radiance, glow and create an even skin tone.

In another aspect, the composition includes niacinamide as a skin lightening agent combined with Pea and Quinoa peptides for enhanced skin effects such as glow, radiance, skin complexion and lightening. Combining the above natural peptides with niacinamide is an exciting new way of delivering skin lightening benefit, enhanced skin glow, radiance, even skin tone and an even skin complexion, reduction of dark spots/hyperpigmentation, and has potential to bring disruption in the market. Products with peptides are incapable of producing unexpected irritations or inflammations. Peptides also work well with many active ingredients without losing effectiveness.

Natural peptides, meaning for this disclosure peptides based on plants, are used in this invention to further these features by removing any constraints that inhibit the features described herein. The combination of natural Pea and Quinoa peptides, and niacinamide assists in delivering a superior result through multi-targeted action. The composition utilizes a combination of three main ingredients in a specific ratio of two natural peptides obtained from hydrolyzed proteins, Pea peptides and Quinoa peptides, with a skin active niacinamide. There are technological challenges as further explained herein when combining these ingredients even though the proteins work well with other ingredients. The invention overcomes these technological challenges to deliver surprising results.

This unique composition provides fast and unexpected results and benefits, immediately enhancing skin glow, even skin tone, skin complexion, skin homogeneity, reducing dark spots and hyperpigmentation, skin radiance within only 7 days and continuing providing effects even after 28 days of treatment. This novel composition also provides multi-targeted biological mechanism of action on skin.

This unique composition is clinically proven to enhance skin glow and radiance and enhance even skin tone, homogeneity, and uniformity of the skin complexion after only 7 and 28 days of treatment. This composition is clinically proven to minimize dark spots/hyperpigmentation and to provide an even skin tone and even complexion in only 7 & 28 days of treatment.

Subjective evaluations results proved that Asian female subjects perceived their skin was glower, more radiant with an even skin tone, reduced dark spots and blemishes, the appearance of skin was glass/porcelain looking like after 28 days of treatment with SG5-42A cream compared to baseline and placebo treatment.

This unique composition can be incorporated in a variety of cosmetic formulations intended to use for face, body and hands use such as creams, lotions, serums, masks, sprays, toners, gels, cream gels, hydrogels, vanishing creams, cold creams, foundations, primers, color cosmetics, balms, cleansers, bath gels, face cleansers and the like.

In one aspect of the invention, between 10-50% niacinamide is used in the formulation, between 10-50% Pea peptides, and 10-50% Quinoa peptides. Less than 100% of the composition includes water and preservative at 1.25%. The preservative may be for example that found in TRI-K's product TriStat E that contains a mixture of Benzyl Alcohol, Water, Potassium Sorbate, and Sodium Benzoate.

Depending on the implementation, the amount of niacinamide may vary in the formulation. Niacinamide may be in a wt. % of 0.001-70%, Quinoa peptides 0.0001%-25%, and Pea peptides 0.0001%-25%. The remaining percentage of the composition, if any, may include, for example, water, and preservative.

In addition, the physical attributes of this composition, include, but are not limited to, light to medium amber aqueous solution, providing a straight addition to any formulation (emulsion, rinse off, leave on, spray), and a highly versatile composition.

The present invention may have also multiple applications, that include but are not limited to, a multifunctional facial, body and hands skin lightening, brightening, glow enhancers, vanishing creams, lotions (O/W emulsions), cold creams, lightening brightening glow anti-aging cosmetic formulations, emulsions, creams, lotions, serums, masks, sprays, toners, gels, foundations, a beauty balm (BB) cream, a color correcting (CC) cream, and a dynamic do-all or daily defense (DD) cream, concealers, make up primers, and the like.

Physical attributes of this composition, includes but are not limited to, the following attributes: light to medium amber aqueous solution, providing straight addition to any formulation (emulsion, rinse off, leave on, spray, O/W, W/O), high versatility, and again does not require pre-solubilization.

Depending on the implementation the ranges again for this novel composition was found to be weight percentages of quinoa (Chenopodium Quinoa) peptides at 0.0001%-25%, pea (Pisum sativum) peptides at 0.0001%-25%, and niacinamide at 0.001-70%. This unique combination was surprisingly found in the clinical studies discussed herein at a total dosage of 0.1-5% to offer benefits to skin glow & radiance, minimize appearance of dark spots & hyperpigmentation, support even skin tone & promote a luminous skin complexion, reduce blotchiness & blurs skin imperfections and attain these visible results in a minimum of 7 days as clinically compared to application of placebos and application of the ingredients individually.

In another aspect of the invention, another example of the preferred embodiment is shown in the Table titled: Skin Care Composition # SG 4-136 Composition Range listed below.

TABLE I
Skin Care Composition # SG 4- 136 Composition Range

| Ingredients Name | | Percentage Range | Percentage Range |
|---|---|---|---|
| Phase A | Supplier | Sample 1 (%) | Sample 2 (%) |
| Water | | Less than | Less than |
| | | 100% | 100% |
| Pea peptides | TRI-K | 10-50% | 10-30% |
| | Industries, Inc | | |
| Quinoa peptides | TRI-K | 1-10% | 5-10% |
| | Industries, Inc | | |
| Niacinamide | DSM | 10-50% | 10-30% |
| Benzyl Alcohol, | TRI-K | 0.5-2% | 1-2% |
| Water, Potassium | Industries, Inc | | |
| Sorbate, Sodium | | | |
| Benzoate | | | |

### Procedure:

1) Add deionized (DI) water to main tank. Start mixing at moderate speed.

2) Add Pea peptides, followed by Quinoa peptides to main tank.

3) Add Niacinamide to ensure that all powder dissolves gradually and does not sink to bottom of the tank.

4) When all powder dissolves, add preservative at the end. Mix until uniform.

These exemplary formulations generate synergistic results as further explained herein. In addition, scientists would not necessarily use the ingredients together that are shown and would not obtain the same results because the specific formula and ratio are both needed. The desired Skin Care composition and method includes a specific ratio of 3 main ingredients Niacinamide, Pea peptides, and Quinoa peptides, and for purposes of this disclosure the term Skin Care composition shall be used interchangeably to mean the above inclusion of said ingredients. These ingredients when combined in the specific ratio do not negatively impact multiple and complex biological pathways of skin melanogenesis, but rather complement each other's mode of action. These ingredients in the specific percentages work synergistically to significantly reduce skin pigmentation and enhance skin glow, radiance, and even skin complexion. These results are clinically proven in vitro 3D skin models as demonstrated herein, in cell cultures and in vivo human skin via clinical efficacy study results and pictures.

There are several technological challenges in combining the three main ingredients of the above composition. In addition, one would not necessarily combine these ingredients together in such an arrangement as proposed. Quinoa peptides are known to denaturize when exposed to heat such as in a chemical reaction, or exposure to ingredients such as niacinamide, vitamin B3, or nicotinic acid. Denatured proteins have a looser, and more random structure. The denaturation of proteins is typically irreversible. A common consequence of denaturation is loss of biological activity. These ingredients would generally not be combined in such a proposed arrangement. In fact, a scientist would most likely not desire to combine these three ingredients in anticipation of an adverse result.

In one aspect, the technological challenges of combining the above ingredients was found to be overcome by mixing the components in the order, conditions, and ratios given herein. It was found that the composition in the specific ratios, as well as combining the order of mixing and conditions actually doesn't produce any negative synergy or side effects.

In addition, after the above ingredients were combined in such a manner, it was surprisingly found that the three main ingredients produced a synergistic effect in the composition. In one aspect, the composition containing the three main ingredients in such a manner provided increase the biological effect as compared to the individual ingredients alone, and provided other properties mentioned above that were not available to the three main ingredients if used individually or with other ingredients.

The above objects and advantages are met by the present invention. In addition, the above and yet other objects and advantages of the present invention will become apparent from the hereinafter-set forth Brief Description of the Drawings, Detailed Description of the Invention and claims appended herewith. These features and other features are described and shown in the following drawings and detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that those having ordinary skill in the art will have a better understanding of how to make and use the disclosed composition and methods, reference is made to the accompanying figures wherein:

FIG. 1. A graphical representation is illustrated of in vitro B16 Melanoma Assay Results. Skin care composition decreased melanin content. Melanin decrease was statistically significant better versus untreated.

FIG. 2. Photomicrographs are shown for skin pigmentation on 3D skin model RHE-MEL. Skin care composition increased the Individual Typology Angle (ITA°) for brightening skin significantly versus untreated and niacinamide benchmarks after 14 days. Skin care composition visibly increased skin glow, radiance, brightness, and even skin tone in the RHE-MEL 3D model.

FIG. 3. A graphical representation is shown of in vitro melanin assay. Skin care composition decreased melanin content in a skin model after 15 days, thus decreasing skin pigmentation for a brighter skin. Melanin decrease was statistically significant better and improved than untreated skin.

FIG. 4. Photomicrographs are shown depicting melanoderma 3D skin pictures. Skin care composition (SG4-136) (right picture) is visibly decreased in melanoderma content after 15 days of treatment. The decreased skin pigmentation for a brighter and lighter in vitro skin appearance is accomplished over the untreated skin (left picture).

FIG. 5. A graphical depiction shows results of skin radiance. SG5-42A cream increased in skin radiance after only 7 and 28 days compared to baseline and placebo cream. The results are statistically significant better compared to baseline and placebo after 7 days and 28 days of treatment.

FIG. 6. A graphical depiction shows the results of a skin glow index study. SG5-42A cream containing skin care composition increased skin glow after only 7 and 28 days. The results are statistically significant better compared to baseline and placebo after 7 days and 28 days of treatment.

FIG. 7. A graphical depiction illustrates SG5-42A cream having increased ITA° and decreased hyperpigmentation and dark spots with an even tone only after 7 and 28 days as compared to a baseline and placebo cream. The results of less visible spots/hyperpigmentation performance of product SG5-42A performed statistical significantly better than placebo and baseline after 7 days and 28 days of treatment.

FIG. 8. A graphical depiction showing SG5-42A effect on dark spots versus normal skin. SG5-42A decreased ΔEa*b* parameter. Skin pigmentation and hue (difference between dark spots and normal skin) showed to be more uniform, homogenous and give even skin ton after only 7 and 28 days. These results are statistically significant better compared to baseline and placebo after 7 days and 28 days of treatment.

FIG. 9. A pictorial illustrates subject evaluation. SG5-42A cream resulted in significantly increased skin glow, radiance, and even skin complexion to reduce dark spots for a glass/porcelain skin appearance compared to a baseline after 28 days of treatment. The sensory performance of product SG5-42A performed better than the placebo after 28 days of treatment.

FIG. 10. Clinical subject photographs illustrate increased skin glow and radiance after treatment with the SG5-42A cream as compared to a placebo. The results of less visible spots/hyperpigmentation performance of product SG5-42A performed better than placebo and baseline after 7 days and 28 days of treatment.

FIG. 11. Clinical subject photographs illustrate skin care composition increased in skin glow and radiance after 7 and 28 days of treatment compared to baseline and placebo skin sites.

### DETAILED DESCRIPTION

The present disclosure is directed to a new composition, and method of making and using a novel combination of natural peptides and niacinamide (Vitamin B3) that is efficacious for skin glow, radiance, skin tone and the like. The present disclosure differs, in one aspect, from current methodologies in that it uses unique natural protein combinations with niacinamide. This combination produced unexpected effects never seen before, and never seen with just using the ingredients of the composition alone. The composition does not further aggravate any side effects of the individual ingredients such as niacinamide that is known to cause skin flushing or tingling.

### Combination of Ingredients

This novel Skin Composition contains two natural peptides, Pea and Quinoa, which work via complementary biological mechanism of action in skin, combined at an optimal ratio with the skin lightening third component of an active niacinamide.

This novel Skin Composition contains natural Pea peptides which were incorporated because it provides unexpected benefits in skin pigmentation and reduces dark spots/hyperpigmentation, increases skin glow, radiance, brightness, luminosity, enhances even skin tone, homogeneity and uniformity of skin complexion, and improves skin's healthy appearance providing overall anti-aging benefits. These results were unexpected because traditionally the incorporation of natural proteins and peptides in cosmetic formulations and their application to skin are generally used to moisturize, hydrate, nourish the skin and improve the overall health of the skin.

In vitro gene expression results clearly demonstrated that the Pea peptides provided a significant effect on skin pigmentation, increasing skin glow, brightness, radiance, reducing dark spots/hyperpigmentation providing an even skin tone and a uniform complexion. The present investigators discovered and demonstrated in gene expression testing assays that this natural Pea peptide component downregulates the expression of nine specific genes (biomarkers) in in vitro 3D Asian skin equivalent models, and inhibits skin melanogenesis multiple biological pathways, reduces skin pigmentation, reduces dark spots/hyperpigmentation, inhibits melanosome transfer and formation, reduces solar lentigines, UV spots, blemishes, red freckles to provide an even skin tone, even complexion and a healthy skin. In addition, this component significantly reduced melanin content in in vitro B16 melanoma cells compared to untreated, showing significant skin lightening, brightening effect in cultured cells.

While conducting and evaluating additional in vitro results obtained from B16 melanoma cultured cells, it was discovered a different and complementary natural peptide such as Quinoa peptides reduced, significantly, the melanin content in B16 melanoma cells, and provided unexpected effects on skin pigmentation, reducing skin pigmentation, increasing skin radiance, glow, lightness.

Quinoa peptides were included in this novel composition as a complementary natural peptide technology, because it provided unexpected results in skin pigmentation, such as reduction of melanin content and inhibition of the melanin transfer from melanocytes to keratinocytes, showing the ability to reduce skin pigmentation and enhance skin luminosity, brightening, glow and enhancing the homogenous aspect of the skin, as well as providing overall anti-aging benefits to skin.

Pea peptides and Quinoa peptides were combined at an optimal ratio with niacinamide as skin lightening active to obtain a novel composition addressing skin pigmentation unmet needs, to enhance skin glow, radiance, brightness, luminosity, lightness and to reduce dark spots/hyperpigmentation and provide an even skin tone, a uniform, homogenous and a healthy skin complexion.

There are different methods of obtaining Pea and Quinoa peptides from protein biomasses. Depending on the embodiment, pea and quinoa peptides may be prepared using hydrolysis via protease or other enzymes, such as, but not limited to, bromelain, chymotrypsin, ficin, papain, amylase, cellulase, trypsin, alkaline proteases and pepsin enzymes, and a combination of those, as well as other techniques. It is known that longer enzyme activity forms more soluble forms. It is known that hydrolysis process can be a function of temperature, time, and pH parameters. Various temperatures may also be utilized to optimize conditions. Different pH values, time, and temperatures ranges may be utilized for the controlled hydrolysis process, depending on the embodiment and may affect the peptide functional properties.

Again, there are different methods of obtaining Pea and Quinoa peptides which are then combined with a skin active, such as niacinamide. The Pea and Quinoa plant peptides processes, as noted above, utilize controlled parameters and conditions to obtain lower average MW distribution between 200-2000 Da. Furthermore, when combining the three main ingredients of the Skin Composition, Pea and Quinoa peptides and niacinamide, whether or not a preservative is present, the pH ideally should be between 4.5-6.0 to avoid any stability issues.

### Biological Mechanism of Action.

This novel composition provides a multi-targeted biological mechanism of action and provides a biological effect across all skin epidermis layers, from the skin surface, stratum corneum and viable epidermis. Each component provides multi-targeted biological mechanism of action on skin and work via complementary mechanisms.

Pea peptides inhibits melanin transfer from melanocytes to keratinocytes, reduces dark spots/hyperpigmentation. Quinoa peptides inhibit melanin transfer from melanocytes to keratinocytes thus reduced skin pigmentation. Niacinamide controls melanin distribution and dispersion in the epidermis at skin surface to enhance skin lightening, brightness and even skin tone.

This unique and optimal combination of two natural peptides Pea peptides and Quinoa peptides with skin lightening active niacinamide provided unexpected results and immediately enhance skin glow, skin radiance, even skin tone, skin complexion, skin homogeneity, uniformity, reduce dark spots and hyperpigmentation, after only 7 days of treatment and providing effects after 28 days of treatment demonstrated during the in vivo clinical studies.

### Description of the In Vitro and In Vivo Clinical Study Results

In Vitro study was conducted on MelanoDerm 3D in vitro Asian model (MatTek) containing normal human derived epidermal keratinocytes and melanocytes. Skin Care Composition (#SG4-136) was evaluated for its ability to decrease melanin content via spectrophotometric analysis (measurement of melanin absorbance units at 403 nm) on 3D MelanoDerm in vitro Asian skin model. Measurements were compared versus untreated skin and performed in triplicates.

In vivo clinical study was conducted on 18 human Asian female volunteers with dark spots/hyperpigmentation on the face and dark skin complexion (Fitzpatrick type IV).

Several measurements were conducted to evaluate the ability of Skin Care Composition (# SG4-136) formulated in a standard vanishing cream (formula SG4-42A) to enhance skin glow, radiance and even skin tone and to reduce dark spots/hyperpigmentation for a homogenous and more uniform skin complexion.

Formula SG4-42A cream (containing Skin Care Composition # SG 4-136) was compared to a placebo cream (SG5-42B) in this single blinded randomized half-face in vivo clinical study.

The below examples are given merely to illustrate the principles of the invention. They do not necessarily limit the scope of the invention to these or any particular embodiments.

### Study 1. In Vitro B16 Melanoma Assay

The in vitro B16 melanoma cells are standard cell cultures models used herein to investigate skin depigmentation effect of biological actives and their effect on melanogenesis. In this in vitro study using B16 melanoma cells, it was proven the synergist effect between the 2 natural peptides for this skin care composition and the synergy action of all 3 complementary ingredients Pea peptides, Quinoa peptides and niacinamide when used at the recorded levels.

In the study B16 melanoma cells were tested using a Spectrophotometric method melanin absorption measured at 405 nm. The B16 melanoma cells were treated with materials for 48 hours. Again, Melanin content was measured via spectrophotometric method (at 405 nm). Measurements were conducted in triplicates. Concentrations were determined based on prescreening MTT assay which determined cell viability. Analysis was done using Student T-test comparison between groups with a (p<0.05). Samples included an Untreated sample, Skin Care Composition at 0.1% (solution), Pea peptides, Quinoa peptides and a Pea+Quinoa peptides combination at corresponding use levels used in Skin Care Composition. Concentrations were determined based on prescreening MTT cytotoxicity assay. MTT assay is an in vitro colorimetric assay for determining cell viability and cytotoxicity activity.

Study results prove that when used alone each natural peptide decrease the melanin content statically significant better compared to untreated control.

However, when Pea and Quinoa peptides are used in a specific optimal ratio, the peptide blend decreased the melanin content in vitro B16 melanoma cells statistically significant better compared to untreated and compared to each peptide alone, thus proving synergy.

When the 2 natural peptides where combined with niacinamide at an optimal ratio, the Skin Care composition (#SG4-136), a combination of three main ingredients in a specific ratio of two natural peptides, Pea peptides and Quinoa peptides, with a skin active niacinamide, described herein that includes decreased the melanin content statistically significant better compared to untreated, each peptide alone, and the combination of the 2 peptides, thus proving synergistic effect due to complementary technologies used in this skin care composition.

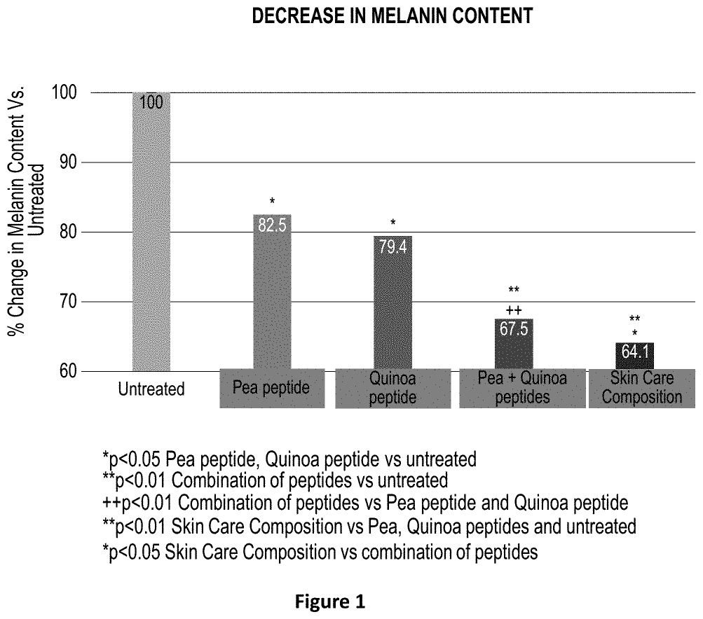

FIG. 1 demonstrates the decrease in melanin content for cells that are untreated, treated only with the pea peptide, treated only with the quinoa peptide, treated with both pea peptide and quinoa peptide, and treated with the Skin Care composition (#SG4-136) that includes all 3 main ingredients Pea peptide, Quinoa peptide and Niacinamide. The cells treated with the Skin Care composition overwhelming had a significant decrease in melanin content as compared to the above other samples tested.

### Study 2. In Vitro Melanin Assay Protocol and Results

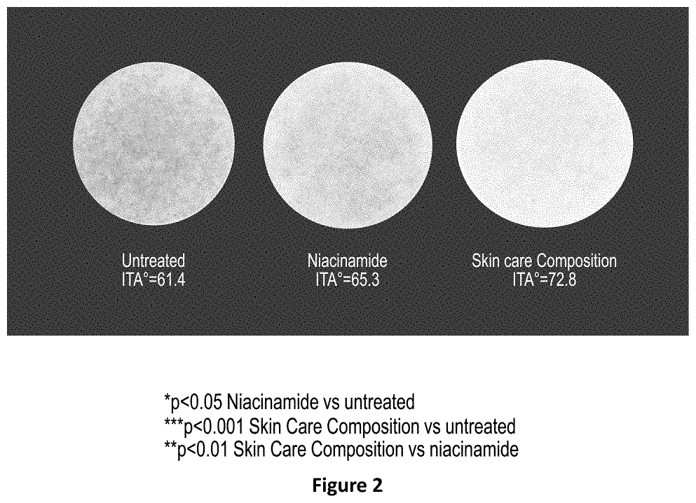

This study demonstrated that skin pigmentation on 3D skin model named RHE-MEL had a statistically significant increased individual typology angle (ITA°) brightening skin than untreated and niacinamide benchmarks after 14 days after treatment. As previously described t calculated ITA° classifies skin types into 7 physiologically different categories: very light, light, intermediate, tan, brown, dark and very dark. According to their ITA°, individuals are assigned into one of the seven skin classification categories. The Skin Care composition with the three ingredients, described above, visibly increased skin glow, radiance, brightness and even skin tone in the RHE-MEL 3d Model as shown in FIG. 2.

The protocol for this study including the following. A melanized reconstructed human epidermis 3D Model (RHE-MEL) (in vitro Caucasian skin model) was used. The study duration was for 14 days. The RHE-MEL 3D in vitro skin tissues were allowed to grow in the first 4 days. The 3D skin tissues were treated every day with the materials for 10 days. The following samples were used: Untreated sample, Benchmark Niacinamide (at same use level as in Skin Care Composition), and Skin Care Composition used at 1%. Concentrations of materials were predetermined based on screening MTT assay. A C-Cube Dermoscope® (a high quality magnifying lens and a powerful lighting system used to evaluate pigmented skin lesions, and can diagnose melanoma), was used coupled with C-Cube clinical software for image analysis. Skin pigmentation was analyzed via high resolution pictures obtained from the C-Cube Dermoscope® coupled with an image analysis software. ITA° parameter was measured as degree of pigmentation of the skin directly on in vitro 3D RHE-MEL skin model. Statistical analysis was done using the Student T-test with a confidence level of (p<0.05).

In this 3D RHE-MEL in vitro Caucasian model skin measured was the intensity of the skin pigmentation degree by ITA° parameter (individual typological angle). Measurements were conducted for the untreated skin, skin treated with Niacinamide (used as benchmark), and skin treated with Skin Care composition after 14 days of treatment. Niacinamide increased the ITA°. The skin pigmentation degree presented significantly better as compared to untreated skin sample. Skin appears lighter, brighter but with an uneven looking appearance after Niacinamide treatment. Skin Care composition treatment to the tissue sample increased the ITA°. The skin pigmentation ITA degree of the Skin Care composition treated tissue sample presented statistically better as compared to untreated and Niacinamide treated tissue samples. The 3D RHE-MEL skin model treated with this Skin Care composition showed increased skin glow, radiance, evenness, and a more uniform and homogeneous appearance compared to untreated and niacinamide treatment.

The complementary technologies from the Skin Care composition such as Pea peptide, Quinoa peptide and Niacinamide are proven to work synergistically to improve skin glow, radiance, even skin tone, homogeneity, smoothness and increase the overall brightness and luminosity of the 3D RHE-MEL in vitro skin model.

### Study 3. In Vitro Melanin Assay Protocol and Results

This composition was tested on melanoderma 3D in vitro Asian skin tissues (MatTek) containing normal human derived epidermal keratinocytes and melanocytes. Melanocytes undergo melanogenesis which leads to melanin accumulation within the tissue overtime. Skin Care composition (SG4-136) was used at 5% solution concentration. The melanoderm tissues were treated with this composition every 24 hours for 15 days. Spectrophotometric measurements of the melanin content (melanin absorbance units at 405 nm) were measured on untreated skin and on skin treated with this composition. Skin tissues were placed in 12 well plate containing 5 ml of agarose assay medium at 37° C. Test materials were applied to the surface of the skin tissues and incubated at 37C. Every 24 hours skin tissues were rinsed with PBS, fresh test material was applied, and the medium was changed every 48 hours. Treatments were carried out for 15 days. Melanin content was measured in the untreated skin tissues and skin tissues treated with Skin Care Composition (SG4-136). Measurements were performed in triplicates. Again, melanin content (mg/ml tissue) was expressed as absorbance measurements at 405 nm. Analysis of Variance (ANOVA) (One-way analysis of variance; p<0.05) was done to compare the results of this testing.

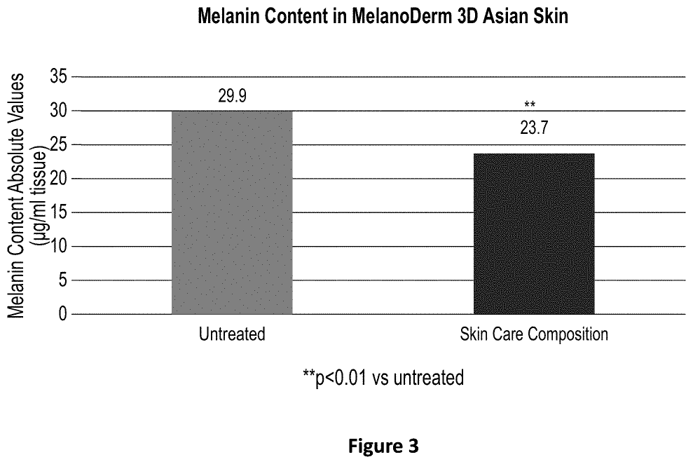

FIG. 3 demonstrates the in vitro study results prove that Skin Care Composition (SG4-136) decreased melanin content in Asian 3 D in vitro skin model after 15 days of use by 21% compared to untreated (statistically significant better compared to untreated).

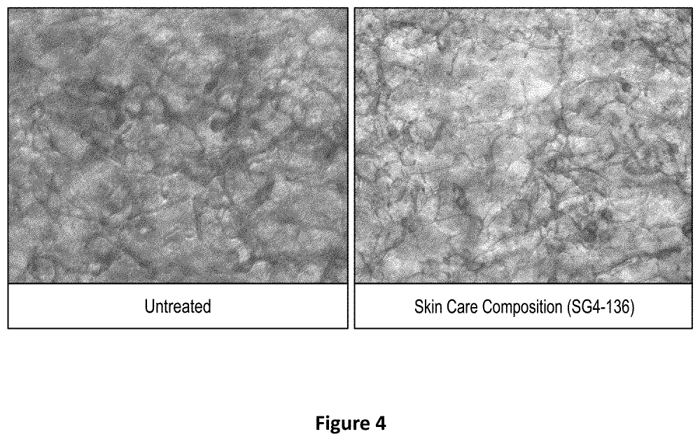

The effect is visible as is clearly illustrated in FIG. 4 as the pictures demonstrated that in vitro skin treated with this composition is visibly brighter and lighter compared to untreated. Skin care composition (# SG4-136) decreased melanin content in Asian 3D in vitro skin model after 15 days, thus decreased skin pigmentation for a brighter skin. Melanin decrease was statistically significant better versus untreated.

### Study 4. In Vivo Clinical Study Protocol and Results

The in vivo clinical study was conducted on 18 Asian (Thai) female volunteers with dark spots, hyperpigmentation, uneven skin tone and dark skin complexion (Fitzpatrick type IV). This was a single blinded half-face randomized type clinical study.

Skin Care Composition Cream (SG5-42A) containing skin care composition (#SG 4-136) at 5% was tested against a placebo cream (SG5-42B) and versus baseline skin conditions for a duration of 28 days.

For this in vivo study the following instrumental measurements were taken at baseline, after 7 days and 28 days of treatment. Complexion grading was assessed on pictures (VISIA® standard light) by trained jury of 3 clinicians. Parameters measured were radiance scores, even complexion scores, skin glow index. Rating scores for these parameters were 0 (worst) to 10 (best). Radiance scores measure the visual perception by human eye of luminosity emanating from the entire skin tone on the face. Rating scale from 0 to 10 where 0 is dull, while and 10 is radiant/luminous.

Even complexion scores measure the uniform aspect of the skin on the face with no visible marks and no change in skin tone. Changes in skin tone (from dark to light) are not considered. Changes in skin redness and rosacea are considered. Rating scale from 0 to 10 where 0 means not even skin, while 10 means very even skin.

Skin glow index measures the sum between radiance scores and even complexion scores. Skin appearance is glass/porcelain like, more radiant, with an even, homogenous, and uniform skin complexion and with a glow and healthier look.

During the same in vivo study, ITA° (Individual typological angle) on dark spots/hyperpigmentation and ΔEa*b* parameters were also measured with a Spectrocolorimeter® CM 700D. Digital pictures were taken with VISIA® instrument at baseline, after 7 days and 28 days of treatment with both products.

ITA° (Individual Typological angle) on dark spots measures the skin pigmentation degree of a subject by measuring L* (luminosity) and b* (cutaneous melanin) components. The higher the ITA°, the lighter and more luminous spots and less hyperpigmentation. An increase in ITA° compared to baseline it means the dark spots are less visible, lighter, and more luminous.

The formula expressed as ΔEa*b* (dark spots−normal skin) measures changes of the skin hue and luminosity. A decrease in ΔEa*b* compared to baseline means an attenuation of pigmentation differences between normal skin and dark spots. This means that dark spots are less visible and less perceivable compared to the normal skin background in near vicinity of the spot, thus skin is more homogenous, uniform with an even tone and an even complexion.

Volunteers were also asked to provide subjective evaluations after 28 days of treatment and to evaluate both products for several sensorial parameters versus baseline and compared to each other.

The in vivo clinical studies conducted on Asian skin female subjects with dark spots, hyperpigmentation, uneven skin tone and dark skin complexion, demonstrated that this skin care composition cream SG5-42A which contains skin care composition (# SG4-136) enhances skin glow, radiance, reduces dark spots/hyperpigmentation, providing an evened skin tone, a homogenous and more uniform skin appearance compared to baseline and placebo, after only 7 days and 28 days of treatment. The in vivo clinical study results, drawings, graphs, and digital pictures are illustrated in FIGS. 4-11.

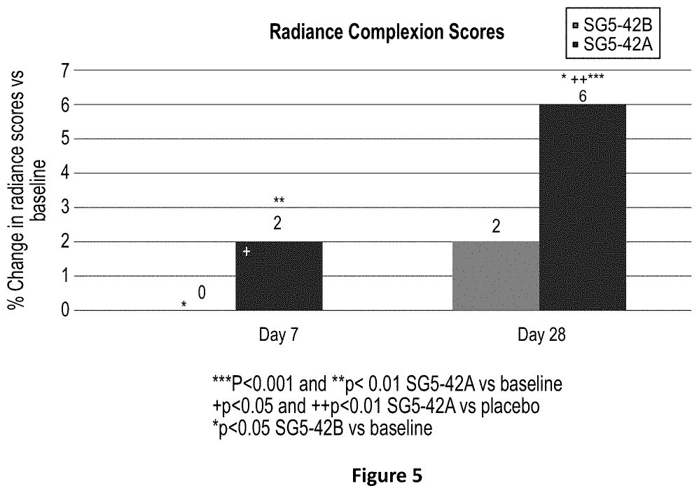

FIG. 5 shows the % change (mean) results of the skin radiance scores after SG5-42A cream treatment versus baseline and SG5-42B placebo cream after 7 and 28 days of treatment. SG5-42A cream which contains Skin Care Composition (#SG4-136) increased by 2% radiance scores versus baseline after 7 days and by 5% radiance scores versus baseline after 28 days of treatment. SG5-42A performed statistically significant better compared to baseline and placebo cream (SG5-42B).

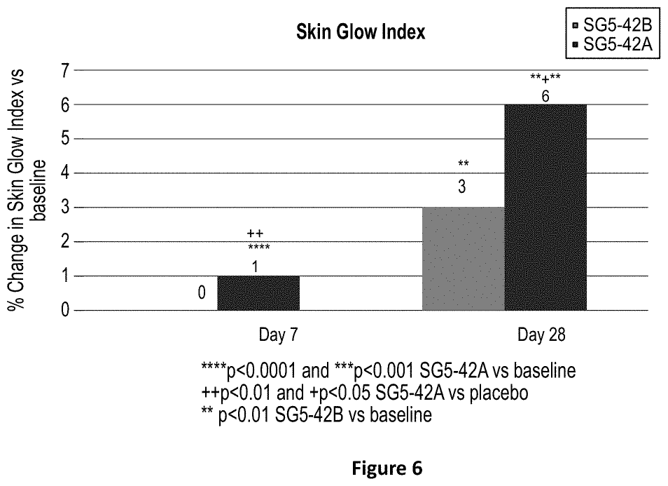

FIG. 6 shows the % change (mean) results of the skin glow index scores after SG5-42A cream treatment versus baseline and placebo cream (SG5-42B). SG5-42A cream increased significantly skin glow by 1% and 5% compared to baseline after 7 days and 28 days of treatment and performed significant better versus placebo cream (# SG5-42B).

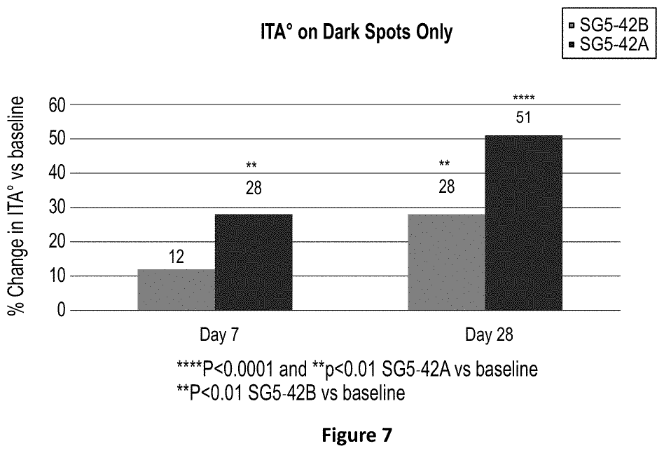

FIG. 7 demonstrates the % change (mean) results of ITA measured on dark spots/hyperpigmentation after SG5-42A cream treatment versus baseline and placebo cream. SG5-42A cream increased significantly the ITA values by 28% after 7 days and by 51% after 28 days of treatment performing significantly better versus baseline and placebo (SG5-42B). Thus SG5-42A decreased the dark spots and hyperpigmentation for less visible dark spots and an even skin tone after 7 days and 28 days of treatment.

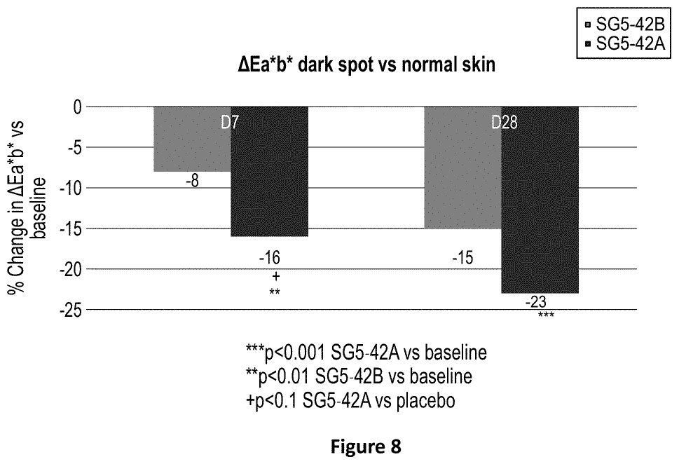

FIG. 8 demonstrates the % change (mean) results of ΔEa*b* parameter (difference between dark spots versus normal skin background) which measures skin homogeneity and even skin tone. SG5-42A cream decreased ΔEa*b* parameter by 16% compared to baseline after 7 days and 23% after 28 days, thus decreased skin pigmentation and hue, creating a more homogenous and even skin tone after 7 days and 28 days of treatment. The results are statistically significant better versus baseline and placebo cream (SG5-42B).

Based on in vivo clinical study results SG5-42A cream increased skin radiance scores and skin glow index after only 7 and 28 days and performed statistically significant better compared to baseline and placebo cream. SG5-42A cream also increased ITA° thus decreased hyperpigmentation and dark spots for less visible lighter spots and an even tone after only 7 and 28 days compared to baseline and placebo cream.

The results are statistically significant better compared to baseline after 7 days and 28 days of treatment.

In Vivo clinical study results demonstrate that formula SG5-42A cream decreased ΔEa*b* parameter, thus skin pigmentation and hue (difference between dark spots and normal skin) for more uniform, homogenous and even skin tone after only 7 and 28 days. The results are statistically significant better compared to baseline and placebo after 7 days and 28 days of treatment.

During subjective evaluations conducted on Asian female volunteers were asked to provide subjective evaluations after 28 days of treatment and to evaluate both products for several sensorial parameters versus baseline and compared to each other.

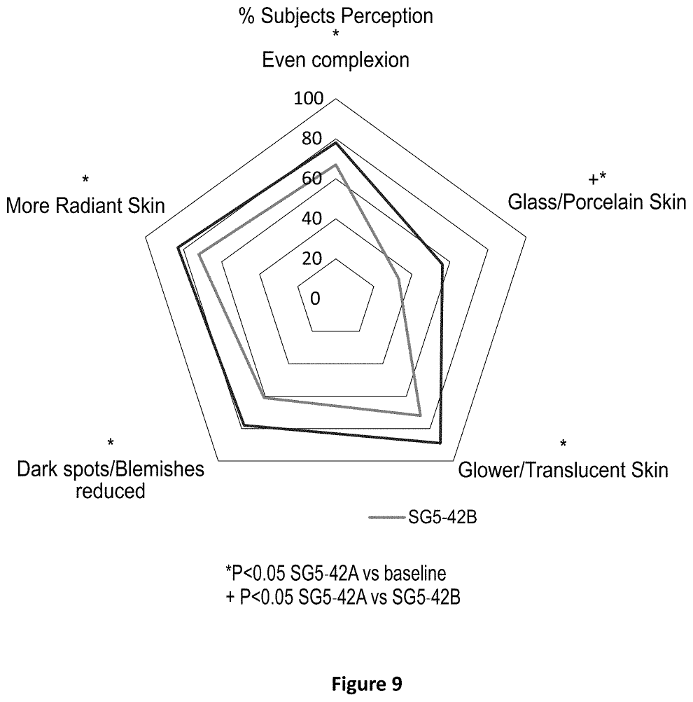

FIG. 9 illustrates % in subjects' perception based on subjective self-evaluation after 28 days of treatment with SG5-42A and SG5-42B compared to baseline.

Subjective evaluation study results demonstrated that after 28 days of treatment with SG5-42A cream versus baseline.

These results indicated the following:
89% of subjects felt that their skin was glower
83% of subjects felt that their skin was more radiant
78% of subjects perceived even skin complexion
78% of subjects perceived that dark spots and blemishes are reduced
56% of subjects perceived a glass/porcelain appearance of the skin

SG5-42A cream was perceived by all subjects to significantly increase the skin glow, radiance, even skin complexion and to reduce dark spots for a glass/porcelain skin appearance compared to baseline after 28 days of treatment.

The sensory performance of product SG5-42A was perceived to perform better than placebo after 28 days of treatment.

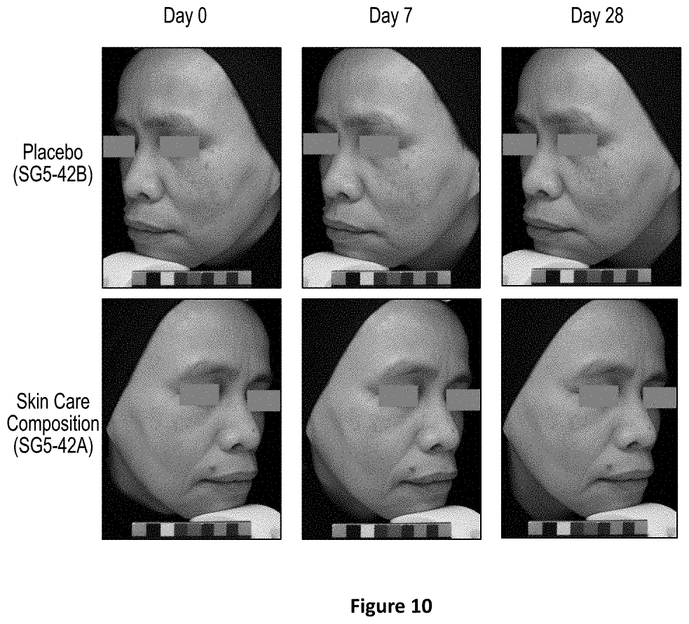

FIG. 10 illustrates several digital pictures for an Asian volunteer (51 years old) taken at Day 0 (baseline) and after 7 days and 28 days of treatment with SG5-42A and B creams. At baseline both skin sites show dark spots, hyperpigmentation, and uneven skin tone with blotches.

After 7 days and 28 days of treatment with SG5-42A cream skin looks radiant, brighter, glower, lighter while dark spots/hyperpigmentation visibly diminished and skin tone is more even, uniform, and homogenous compared to baseline and placebo skin sites.

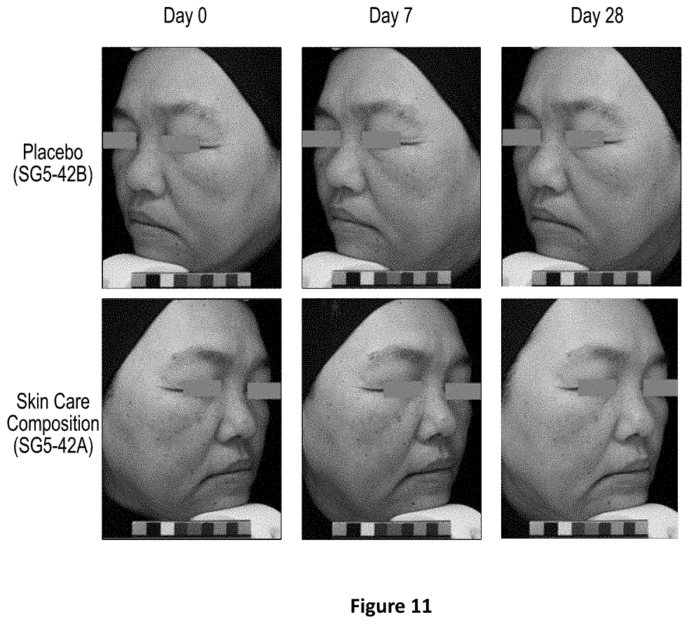

FIG. 11 illustrates several digital pictures for a volunteer test subject using the Skin Care composition and reviewed after 7 and 28 days of treatment. Shown in FIG. 11 was an increase in the test subject's skin glow and radiance as measured and previously described. For this test subject, Skin Care Composition increased skin glow by 14%, skin radiance by 17%, and skin evenness by 23% after 28 days of treatment. After 7 and 28 days of treatment with the Skin Care Composition, the test subject's skin looks visibly more radiant, glows more than untreated test subject's skin, while dark spots/hyperpigmentation are visibly diminished and skin tone is more even, uniform, and homogenous as compared to baseline and placebo skin sites.

### In Vivo Clinical Study Summary of Results

Based on in vivo clinical study results SG5-42A cream increased skin radiance scores and skin glow index after only 7 and 28 days and performed statistically significant better compared to baseline and placebo cream.

SG5-42A cream also increased ITA° thus decreased hyperpigmentation and dark spots for less visible lighter spots and an even tone after only 7 and 28 days compared to baseline and placebo cream. The results are statistically significant better compared to baseline after 7 days and 28 days of treatment.

In Vivo clinical study results demonstrate that formula SG5-42A cream decreased ΔEa*b* parameter, thus skin pigmentation and hue (difference between dark spots and normal skin) for more uniform, homogenous and even skin tone after only 7 and 28 days.

The results are statistically significant better compared to baseline and placebo after 7 days and 28 days of treatment.

SG5-42A cream was perceived by all subjects to significantly increase the skin glow, radiance, even skin complexion and to reduce dark spots for a glass/porcelain skin appearance compared to baseline after 28 days of treatment.

The sensory performance of product SG5-42A was perceived to perform better than placebo after 28 days of treatment.

The results are visible and clearly demonstrated via digital pictures for a representative subject (age 51 years old).

This Asian volunteer (51 years old) showing at Day 0 (baseline) dark spots, hyperpigmentation, and uneven skin tone.

After only 7 days and 28 days of treatment with SG5-42A cream skin looks radiant, brighter, glower, lighter while dark spots/hyperpigmentation visibly diminished and skin tone is more even, uniform, and homogenous compared to baseline and placebo skin sites.

TABLE II
Formulation Composition # SG5-42A (with Skin Care Composition SG4-
136) and SG5-42B (placebo) used for the in vivo clinical study

| | | | Percentage | |
|---|---|---|---|---|
| | | | (%) SG5-42A | |
| | Example | | (With Skin | Percentage |
| | Commercial | Example | Care | (%) SG5-42B |
| Ingredient Name | Name | Supplier | Composition) | (Placebo) |
| Phase A | | | | |
| Water | Water | | 66.456 | 71.456 |
| Glycerin | Glycerin 99.5% | Protameen | 1.00 | 1.00 |
| | Natural USP | Chemicals | | |
| Tetrasodium Glutamate Diacetate | BioPure ™ GLDA | Jarchem | 0.04 | 0.04 |
| | | Industries, | | |
| | | Inc. | | |
| Phase B | | | | |
| Stearic Acid | Stearic Acid TP | Jeen Intl. | 17.90 | 17.90 |
| | NF | Corp. | | |
| Cetyl Alcohol | Cetyl Alcohol NF | Jeen Intl. | 0.53 | 0.53 |
| | | Corp. | | |
| Hydrogenated Ethylhexyl | Fision ® EcoSil | TRI-K | 0.50 | 0.50 |
| Olivate, Hydrogenated | | Industries, | | |
| Olive Oil Unsaponifiables | | Inc. | | |
| Isopropyl Myristate | Jeechol IPM | Jeen Intl. | 0.75 | 0.75 |
| | | Corp. | | |
| Titanium Dioxide, Aluminum | TRIShield T-110 | TRI-K | 0.20 | 0.20 |
| Hydroxide, Stearic Acid | | Industries, | | |
| | | Inc. | | |
| Tocopheryl Acetate | Vitamin E | TRI-K | 0.10 | 0.10 |
| | Acetate | Industries, | | |
| | | Inc. | | |
| Ethylhexyl Methoxycinnamate | Galaxy 2932 | Galaxy | 0.75 | 0.75 |
| | | Surfactants | | |
| | | Ltd. | | |
| Butyl Methoxydibenzoylmethane | Jeescreen A | Jeen Intl. | 0.40 | 0.40 |
| | | Corp. | | |
| Phase C | | | | |
| Water | Water | | 5.00 | 5.00 |
| Potassium Hydroxide | Potassium | J. T. Baker | 0.484 | 0.484 |
| | Hydroxide, | | | |
| | Pellets | | | |
| Phase D | | | | |
| Water, Niacinamide, Pea | Skin Care | TRI-K | 5.00 | 0.00 |
| Peptides, Quinoa peptides | Composition | Industries, | | |
| | (#SG4-136) | Inc. | | |
| Phase E | | | | |
| Phenoxyethanol | TRIstat P5 | TRI-K | 0.89 | 0.89 |
| | | Industries, | | |
| | | Inc. | | |

### Procedure:

1) Heat water to 85° C. and add Phase A ingredients one at a time until dissolve and uniform.

2) Combine Phase B with heat and mixing to 85° C. Combine Phase C.

3) Transfer Phase B to Phase A to emulsify and switch to homogenizer.

4) Do not let temperature drop. Keep at 85° C. Add Phase C under homogenizer.

5) Switch to side sweep mixing. Cool to 40° C. Add Phase D and Phase E.

### Skin Care Composition: Summary of Skin Benefits and Claims

This novel composition contains two natural peptides Pea and Quinoa, which work via complementary biological mechanism of action in skin, and are combined at an optimal ratio with the skin lightening active Vitamin B3 (Niacinamide). This optimal combination of Vitamin B3 (Niacinamide) and complementary natural Pea and Quinoa peptides can offer a holistic approach to the skin glow through multi targeted action controlling key biological pathways to deliver the desired end benefit of enhancing skin glow, skin radiance and even skin tone and even skin complexion.

This unique and optimal combination of two natural peptides Pea and Quinoa with the skin lightening active niacinamide provided unexpected results and immediately enhance skin glow, skin radiance, even skin tone, skin complexion, skin homogeneity, uniformity, reduce dark spots and hyperpigmentation, after only 7 days of treatment and providing effects after 28 days of treatment demonstrated during the in vivo clinical studies.

This novel composition provides a multi-targeted biological mechanism of action and provides a biological effect across all skin epidermis layers, from the skin surface, stratum corneum and viable epidermis. Each component provides multi-targeted biological mechanism of action on skin and work via complementary mechanisms. Pea peptides inhibit melanin transfer from melanocytes to keratinocytes, reduces dark spots/hyperpigmentation. Pea peptides inhibit melanin transfer from melanocytes to keratinocytes, reduces dark spots/hyperpigmentation. Quinoa peptides inhibit melanin transfer from melanocytes to keratinocytes thus reduced skin pigmentation. Niacinamide controls melanin distribution and dispersion in the epidermis at skin surface to enhance skin lightening, brightness and even skin tone.

### Summary of Results

Below is a brief summary of the results using this novel composition. Again these embodiments are given to merely illustrate the principles of the invention and are not meant to limit the invention to these particular examples.

Pea peptides inhibit melanin transfer from melanocytes to keratinocytes, reduces dark spots/hyperpigmentation. Quinoa peptides inhibit melanin transfer from melanocytes to keratinocytes thus reduce skin pigmentation. Niacinamide controls melanin distribution and dispersion in the epidermis at skin surface to enhance skin lightening, brightness and even skin tone.

Clinically proven to enhance skin glow and radiance after only 7 and 28 days of treatment.

Clinically proven to enhance even skin tone, homogeneity, and uniformity of the skin complexion after only 7 days and 28 days of treatment.

Clinically proven to minimize dark spots/hyperpigmentation and to provide an even skin tone and even complexion in only 7 & 28 days of treatment.

Subjective evaluations results proved that Asian female subjects perceived their skin was glower, more radiant with an even skin tone, reduced dark spots and blemishes, the appearance of skin was glass/porcelain looking like after 28 days of treatment with SG5-42A cream compared to baseline and placebo treatment.

Digital pictures demonstrate visible improvements for a representative Asian volunteer (51 years old) which shows at Day 0 (baseline) dark spots, hyperpigmentation, and uneven skin tone. After 7 days and 28 days of treatment with SG5-42A cream skin looks more radiant, brighter, glower, while dark spots/hyperpigmentation are visibly diminished, while skin tone is more even, uniform, and homogenous compared to baseline and placebo skin sites.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claims is:

1. A skin care composition for skin glow, radiance, and skin tone, comprising the following components:
a first component having an effective amount by weight of quinoa (chenopodium quinoa) peptides in a range of 0.0001%-25%;
a second component having an effective amount by weight of pea (pisum sativum) peptides in a range of 0.0001%-50%; and
a third component having an effective amount by weight of niacinamide in a range of 0.001-70%, wherein the first, the second, and the third components form a composition that has an effect on an epidermis of a user and lightens the epidermis for skin glow, radiance, and skin tone.

2. The composition of claim 1, wherein the effective amount of niacinamide is between 10-50% by weight, the effective amount of pea peptides is 10-50% by weight, and the effective amount of quinoa peptides is 1-10% by weight.

3. The composition of claim 2, further including an amount by weight of water, and a preservative.

4. The composition of claim 3, wherein the amount of water by weight is less than 100%, and the preservative is 0.5-2%.

5. The composition of claim 4, wherein the preservative includes Benzyl Alcohol, Water, Potassium Sorbate, and Sodium Benzoate.

6. The composition of claim 1, wherein the effect on the epidermis is immediately after application of the composition and enhancement of skin glow, skin radiance, even skin tone, skin complexion, skin homogeneity, uniformity, reduction of dark spots; and hyper-pigmentation is visibly observed after only 7 days of treatment and the composition continues to provide the effect after 28 days of treatment.

7. The composition of claim 1, wherein each component provides multi-targeted biological mechanism of action on skin and work via complementary synergistic mechanisms; and
the effective amount of Pea peptides inhibit melanin transfer from melanocytes to keratinocytes, reduces dark spots/hyperpigmentation;
the effective amount of Quinoa peptides inhibit melanin transfer from melanocytes to keratinocytes and reduces skin pigmentation; and
the effective amount of the niacinamide controls melanin distribution and dispersion in the epidermis at its surface to enhance skin lightening, brightness, and even skin tone.

8. The composition of claim 1, wherein the composition provides a multi-targeted biological mechanism of action and provides a biological effect across all of the epidermis layers, from skin surface, stratum corneum, and viable epidermis.

9. The composition of claim 1, wherein the first component, the second component, and the third component combined is clinically proven in in vitro skin models and in vivo human skin to enhance skin glow, radiance, even skin tone, homogeneity, uniformity, and smooth skin texture to improve skin appearance after only 7 days and 28 days of treatment as compared to the components individually, or only two components combined.

10. The composition of claim 1, wherein the first, the second, and the third components are incorporated in a cosmetic formulation for face, body, and hands, and the cosmetic formulation is selected from a group consisting of a cream, a lotion, a serum, a mask, a spray, a toner, a gel, a cream gel, a hydrogel, a vanishing cream, a cold cream, a foundation, a primer, a color cosmetic, a balm, a cleanser, a bath gel, a face cleanser, and any combination thereof.

11. The composition of claim 1, further including a fourth component of water less than 70% by weight to form a Phase D.

12. The composition of claim 11, further including a Phase A, a Phase B, a Phase C, and a Phase E, and further comprises:
the Phase A having water, glycerin and tetrasodium glutamate diacetate;
the Phase B having stearic acid, cetyl alcohol, hydrogenated ethylhexyl olivate, hydrogenated olive oil, unsaponifiables, isopropyl myristate, titanium dioxide, aluminum hydroxide, stearic acid, tocopheryl acetate, ethylhexyl methoxycinnamae, and butyl methoxydibenzoylmethane;
the Phase C having water and potassium hydroxide; and
the Phase E having phenoxyethanol.

13. The composition of claim 12, wherein the Phase A is between 0.1-67%, the Phase B is between 0.1-20% the Phase C is between 0.1-5.5%, the Phase D is between 0.1-10%, and the Phase E is between 0.1-1%.

14. The composition of claim 13, wherein the Phase D is 5%.

15. A method of using a composition for skin glow, radiance and even skin tone comprising:
topically applying to an epidermis of a user a composition that includes a first component having an effective amount by weight of Quinoa peptides in a range of 0.0001%-25%, a second component having an effective amount by weight of Pea peptides in a range of 0.0001%-25%, and a third component having an effective amount of niacinamide in a range of 0.001-70%, wherein the composition lightens the epidermis of a user for skin glow, radiance and even skin tone.

16. The method of claim 15, wherein the topically applying step further includes applying the composition once a day continuously for at least 7 days for visible observation of an effect on the epidermis after application of the composition and enhancement of skin glow, skin radiance, even skin tone, skin complexion, skin homogeneity, uniformity, reduction of dark spots, and hyperpigmentation.

17. The method of claim 16, further includes using the composition as multifunctional facial, body, and hands cosmetic selected from a group consisting of a skin lightening, a skin brightener, a glow enhancers, a vanishing cream, an oil in water (O/W) emulsion, a cold cream, a lightening brightening glow anti-aging cosmetic formulation, an emulsion, a cream, a lotion, a serum, a mask, a spray, a toner, a gel, a foundation, a beauty balm (BB) cream, a color correcting (CC) cream, and a dynamic do-all or daily defense (DD) cream, a concealer, a make-up primer, and any combination thereof.

18. A method of making a skin care composition for skin glow, radiance, and skin tone, comprising:
adding deionized (DI) water into a main tank, and mixing;
adding to the main tank a second component having an effective amount by weight of Pea peptides in a range of 0.0001%-25%, followed adding by a first component having an effective amount by weight of Quinoa peptides in a range of 0.0001%-25%.
adding into the main tank a third component having an effective amount of niacinamide in a range of 0.001-70% and dissolving all components gradually so that none of the components sink to bottom of the tank to form a composition; and
wherein the composition lightens an epidermis of a user for skin glow, radiance and even skin tone.

19. The method of claim 18, further includes adding into the main tank a preservative having an effective amount of phenoxyethanol in a range 0.1-1% after all the components dissolve and mixing the preservative until the preservative is uniformly dispersed to for a mixture.

20. The method of claim 19, further includes maintaining the mixture at a pH from 6.0-8.0.
